(19) United States
(12) Patent Application Publication
(10) Pub. No.: US 2006/0111295 A1
(43) Pub. Date: May 25, 2006

(54) Cellular receptors utilized as carrier agents for pharmaceutical compounds used in the treatment of arthritis, inflammation and immune disorders

(76) Inventors: Henry J. Smith, Temecula, CA (US); James R. Smith, Laguna Niguel, CA (US)

Correspondence Address: STETINA BRUNDA GARRED & BRUCKER, 75 ENTERPRISE, SUITE 250, ALISO VIEJO, CA, 92656, US

(21) Appl. No.: 11/284,573

(22) Filed: Nov. 22, 2005

Related U.S. Application Data
(60) Provisional application No. 60/629,918, filed on Nov. 22, 2004.

(51) Int. Cl. A61K 38/17

(52) U.S. Cl. 514/12

(57) ABSTRACT

This invention describes a method of utilizing soluble receptors such as tumor necrosis factor receptor or interleukin receptor to carry pharmaceutical compounds to areas of inflammation. Patients with inflammatory disease such as arthritis, or cardiomyopathy or other inflammatory conditions may benefit from this treatment.

### CROSS-REFERENCE TO RELATED APPLICATIONS

The present application is a non-provisional application claiming priority to U.S. Provisional Patent Application Ser. No. 60/629,918 entitled CELLULAR RECEPTORS UTILIZED AS CARRIER AGENTS FOR PHARMACEUTICAL COMPOUNDS USED IN THE TREATMENT OF ARTHRITIS, INFLAMMATION AND IMMUNE DISORDERS, filed Nov. 22, 2004.

### STATEMENT RE: FEDERALLY SPONSORED RESEARCH/DEVELOPMENT

Not Applicable

### BACKGROUND

The main application of this invention is in the treatment of immunological disorders such as rheumatoid arthritis and other inflammatory conditions. A common symptom of rheumatoid arthritis is swollen, painful joints. For mild cases of arthritis treatment usually consists of aspirin or non-steroidal anti-inflammatory drugs. For more severe cases steroidal drugs such as cortisone, prednisone and methylprednisolone are used. Finally, in cases where the patients become non-responsive to these drugs, more cytotoxic drugs such as methotrexate may be used. In addition to their therapeutic effect, these drugs all have a systemic effect and can cause serious side-reactions. It is desirable to have a treatment process that would be more effective upon the disease with less side effects.

This invention teaches a method whereby the effect of these drugs may be enhanced by combining them with a specific carrier protein to cause them to concentrate at the site of inflammation where they can have the most effect and also to mitigate their undesirable systemic side effects.

The novelty of this invention lies in the use of cellular receptors as carriers of pharmaceutical compounds. This teaching is the reverse of what is normally taught to occur in the body. In the body, cells communicate with each other via a large variety of chemical messengers. For example messengers such as hormones, growth factors and cytokines circulate in the body until they reach their target cells where they will bind to their specific receptors on the cell. Under normal circumstances, the messenger is the mobile entity and the cellular receptor that it targets is the immobile entity being fixed to the cell membrane. However, this invention teaches of a reverse process whereby there are means of making cellular receptors that are soluble and not attached to a cell; and that there are situations where localized inflamed areas in the body may have elevated concentrations of messengers being secreted by cells; and that these inflamed areas can be targeted using said soluble cellular receptors. The soluble receptors will circulate until they reach these areas where they will bind most to areas where their messengers have the highest concentration and thus become fixed to that area. By combining pharmaceutical compounds to the soluble receptors, the pharmaceuticals can be selectively transported to these areas where they will have the most therapeutic effect.

Arthritis and other inflammatory diseases often have localized areas of inflammation where elevated levels of inflammatory messenger proteins may be found. For example, the inflamed areas in arthritic joints may have elevated levels of cytokines such as tumor necrosis factor (TNF) and/or interleukins (IL). By using soluble tumor necrosis factor receptor (TNFR) labeled with an anti-inflammatory drug, or soluble interleukin receptor (ILR) labeled with an anti-inflammatory drug, it is possible to deliver the drug to the inflammatory site where it will have the most effect.

This method can also be used to treat patients with other immune disorders that have an inflammatory condition. For example, patients with cardiomyopathy have an inflammatory condition of the heart. By using soluble tumor necrosis factor receptor (TNFR) labeled with an anti-inflammatory drug, or soluble interleukin receptor (ILR) labeled with an anti-inflammatory drug, it is possible to deliver the drug to the inflammatory site where it will have the most effect.

This method can also be used to treat patients with other immune disorders that have an inflammatory condition. For example, patients receiving an organ or tissue transplant may develop an inflammatory reaction to the graft. By using soluble tumor necrosis factor receptor (TNFR) labeled with an anti-inflammatory drug, or soluble interleukin receptor (ILR) labeled with an anti-inflammatory drug, it is possible to deliver the drug to the inflammatory site where it will have the most effect.

A further benefit of this invention is that because the cellular receptors are human proteins, they are non-immunogenic and will not elicit an immune response by the patient. They can therefore be used repeatedly as “carriers” for anti-inflammatory drugs over a prolonged period of time.

### BRIEF SUMMARY

This invention describes the use of soluble cellular receptors as carrier agents for pharmaceutical compounds used to treat various immune disorders. The labeled carrier compounds have the propensity of binding to substances found in areas of tissue damage associated with many disease conditions such as those found in inflammatory immune disorders. By combining different drugs to these carrier proteins, it is possible to develop a variety of new pharmaceuticals to treat arthritis and other immune disorders.

The soluble receptors are derived from human cells and are therefore non-immunogenic to the human patient. They can be used repeatedly over a period of time without evoking an adverse immune response in the patient.

### DETAILED DESCRIPTION

This invention teaches a method for improved delivery of pharmaceutical agents to a target tissue site. The target tissue may be an inflammatory site within an affected tissue or organ. The invention describes the process of obtaining isolated soluble cellular receptors, and the process whereby various therapeutic agents are combined with soluble cellular receptors and used in the treatment of arthritis and immune disorders. The receptors have a propensity for binding to certain substances occurring in areas of inflammation and will in turn cause the thereapeutic compounds linked to them to localize within the affected tissue areas where they will have the maximum therapeutic effect.

In the preferred embodiments of this invention, the cellular receptors used are from the tumor necrosis factor family group of receptors (TNF-R); and from the interleukin family group of receptors (IL-R). However, it will be obvious to those skilled in the art that other soluble cellular receptors may be similarly employed and these are therefore included within the scope of this invention.

In this invention, the term “tumor necrosis factor receptor” or “(TNF-R)” refers to all the members of the family of tumor necrosis factor receptors. The term also refers to the binding moieties of the receptor molecule whether these are the free binding fragments and/or the binding fragments combined with other compounds such as part of a recombinant fusion protein, or fixed to a protein or to a peptide by adsorption or chemical process.

This invention describes the use of tumor necrosis factor receptor (TNF-R) as a carrier protein for pharmaceutical drugs. Tumor necrosis factor is a cytokine that is produced by a variety of cells involved in the immune response. It is secreted into the extra-cellular medium and binds to other inflammatory cells that have tumor necrosis factor receptors on the cell membrane causing these cells to participate in the inflammatory response.

The tumor necrosis factor receptors can be isolated from the cellular membrane by standard laboratory techniques. For example, cells are homogenized and the cell membranes isolated by differential certifugation. The cell membranes are solubilised using a variety of detergent solutions and the soluble receptors are then purified using gel-chromatography, or high pressure liquid phase chromatography, or other standard laboratory techniques.

Tumor necrosis factor receptors can also be prepared as a recombinant protein using genetic engineering techniques. For example, the genetic code for TNF-R is cloned using the polymerase chain reaction and attached to plasmid DNA. The altered plasmid DNA is used to transform E. Coli bacteria which are grown in fermentation tanks. The transformed bacteria produce human TNF-R which is purified using standard methods such as ion exchange chromatography, and/or gel permeation and reverse-phase chromatography. The recombinant TNF-R may be expressed either complete, or as a fragment which has TNF binding capacity, or as part of a recombinant fusion protein. In this context, TNF-R refers to either the complete tumor necrosis factor receptor, or the binding fragment of TNF-R, or TNF-R as a component of a fusion protein molecule. The recombinant TNF-R can also be produced using other recombinant protein expression systems such as yeast cells or insect cells or mammalian cells, without affecting the novelty of this invention.

In this invention, the term “interleukin receptor” or “(IL-R)” refers to all the members of the family of interleukin factor receptors. The term also refers to the binding moieties of the receptor molecule whether these are the free binding fragments and/or the binding fragments combined with other compounds such as part of a recombinant fusion protein, or fixed to a protein or to a peptide by adsorption or chemical process.

This invention describes the use of interleukin receptor (IL-R) as a carrier protein for pharmaceutical drugs. There are a number of different interleukins secreted by different types of cells. The cells secrete the interleukin into the extracellular medium where it binds to other inflammatory cells that have interleukin receptors on the cell membrane causing these cells to participate in the inflammatory response.

The interleukin receptors can be isolated from the cellular membrane by standard laboratory techniques. For example, cells are homogenized and the cell membranes isolated by differential certifugation. The cell membranes are solubilised using a variety of detergent solutions and the soluble receptors are then purified using gel-chromatography, or high pressure liquid phase chromatography, or other standard laboratory techniques.

Interleukin receptors can also be prepared as a recombinant protein using genetic engineering techniques. For example, the genetic code for IL-R is cloned using the polymerase chain reaction and attached to plasmid DNA. The altered plasmid DNA is used to transform E. Coli bacteria which are grown in fermentation tanks. The transformed bacteria produce human IL-R which is purified using gel-chromatography, or high pressure liquid phase chromatography, or other standard laboratory techniques.

Interleukin receptors can also be prepared as a recombinant protein using genetic engineering techniques. For example, the genetic code for IL-R is cloned using the polymerase chain reaction and attached to plasmid DNA. The altered plasmid DNA is used to transform E. Coli bacteria which are grown in fermentation tanks. The transformed bacteria produce human IL-R which is purified using standard methods such as ion exchange chromatography, and/or gel permeation and reverse-phase chromatography. The recombinant IL-R may be expressed either complete, or as a fragment which has interleukin binding capacity, or as part of a recombinant fusion protein. In this context, IL-R refers to either the complete interleukin receptor, or the binding fragment of IL-R, or IL-R as a component of a fusion protein molecule. The recombinant IL-R can also be produced using other recombinant protein expression systems such as yeast cells or insect cells or mammalian cells, without affecting the novelty of this invention.

There are a large variety of pharmaceuticals that can be attached to the receptor molecule and used to treat arthritis and other inflammatory conditions. In addition to the cytotoxic drugs such as methotrexate, there are the recently developed immune modulating drugs such as cyclosporine and rapamycin and these can also be attached to the receptor molecule and transported to the inflamed site. There are various ways of conjugating a pharmaceutical compound to the soluble receptor. Amino groups cross-link with N-succinimydalcarboxylates and this is energetically favorable in a thiol-free environment. Amino groups can also react with aldehyde groups, which forms an unstable double bond and one of them reduces to yield a stable single bond cross-link. In addition, if the amino group reacts with glutaraldehyde, it forms a stable cross-link without the extra step. Amino groups can also react with acid anhydrides, yielding a cross-linked product and a carboxylic acid. Amino groups can react with para-benzoquinone at two sites depending on the pH. At a high pH, amino groups will react with isothiocyanates. Having a label containing a thiol allows reaction with malemeide groups. Thiol groups can also react with pyridylsulfide groups. The methods of conjugating any particular drug to the carrier protein will vary depending upon the nature of the drug. However, these are according to conventional laboratory methods and are considered to be within the scope of this invention.

The pharmaceutical labeled receptor can be used to treat arthritis patients or those with other inflammatory conditions. In the preferred embodiment of this invention it may be preferable to use receptor binding fragments instead of the whole molecule as the carrier. These will be of smaller size than the complete receptor molecule and therefore will penetrate more easily into the inflammatory areas such as the affected joints in arthritic patients.

In certain circumstances, it may be preferred to divide the treatment into two stages. This is because patients with an inflammatory condition will have cytokines generally distributed in the circulation and in the extravascular space. Therefore when patients are treated with a pharmaceutical labeled receptor there will be immediate binding of the receptor to the circulating cytokines before there is penetration of the labeled receptor into the inflammatory area. The first stage therefore is to treat the patient with soluble receptor alone to bind out the circulating cytokines, followed by the second stage which is to treat the patient with the pharmaceutical labeled soluble receptor.

Alternatively, the first stage can be to treat the patient's blood using apheresis. Apheresis is a procedure whereby the patient's blood is passed through an extracorporeal device that removes the circulating cytokines by binding them to immobilized receptors and then returning the cleaned blood back into the patient. The immobilized receptors to tumor necrosis factor or to interleukins or other cytokines are prepared by chemically coupling them to an insoluble support matrix. For example tumor necrosis factor receptor can be conjugated to activated sepharose beads using the cyanogen bromide method. Other methods of immobilizing the receptors and preparing affinity devices are known to those skilled in the art and are within the scope of this invention.

This invention describes a process for treating a variety of inflammatory conditions including arthritis, cardiomyopathy, and transplant patients. Other inflammatory diseases may similarly be treated and are within the scope of this invention.

As the carrier receptors are non-immunogenic, they can be used repeatedly over a period of time without provoking a host immune response from the patient.

This invention is not limited to the examples described herein. It will be obvious to those skilled in the art that other types of soluble receptors can be used as carrier proteins for a variety of pharmaceutical compounds. However, they will all utilize the principles described in this invention of using soluble receptors as a carrier agent to deliver a particular drug to the site of inflammation, and therefore fall within the scope of this invention.

What is claimed is:

1. A process to treat arthritis and other inflammatory disorders using soluble receptors as a carrier agent to deliver anti-inflammatory compounds to the site of inflammation.

2. A process according to claim 1 whereby the receptor is tumor necrosis factor receptor (TNF-R).

3. A process according to claims 1 and 2 where the term “tumor necrosis factor receptor” or “TNF-R” includes all the members of the family of tumor necrosis factor receptors.

4. A process according to claims 1-3 where the term “tumor necrosis factor receptor” or “TNF-R” includes the whole tumor necrosis factor receptor molecule; and/or the ligand binding sites of fragments of the tumor necrosis factor receptor molecule; and/or the ligand binding sites of the tumor necrosis factor receptor fragment when it is part of a recombinant fusion protein.

5. A process according to claim 1 whereby the receptor is interleukin receptor (IL-R).

6. A process according to claims 1 and 5 where the term “interleukin receptor” or “IL-R” includes all the members of the family of interleukin receptors.

7. A process according to claims 1, 5 and 6 where the term “interleukin receptor” or “IL-R” includes the whole interleukin receptor molecule; and/or the ligand binding sites of fragments of the interleukin receptor molecule; and/or the ligand binding sites of the interleukin receptor fragment when it is part of a recombinant fusion protein.

8. A process according to claim 1 whereby the soluble receptor is a cytokine binding cellular receptor.

9. A process according to claims 1 and 8 where the term “cytokine binding cellular receptor” includes the whole cytokine binding receptor molecule; and/or the ligand binding sites of fragments of the cytokine receptor molecule; and/or the ligand binding sites of the cytokine receptor fragment when it is part of a recombinant fusion protein.

10. A process according to claim 1 of combining anti-inflammatory pharmaceuticals to the soluble receptor.

11. A process according to claim 1 of combining immune modulating pharmaceuticals to the soluble receptor.

12. A process according to claim 1 that uses a two-stage process in which the first stage is to use an unlabeled receptor to bind out the circulating cytokines, followed by the second stage in which the patient receives the pharmaceutical-labeled receptor.

13. A process according to claim 1 that uses a two stage process in which the first uses an apheresis process to bind out circulating cytokines, followed by the second stage in which the patient receives the pharmaceutical-labeled receptor.

14. A process according to claim 1 whereby the use of soluble human receptors as a carrier agent for pharmacological products will not elicit an antibody response by the patient to the receptor carrier protein.

15. A process according to claim 1 whereby the patient can receive repeated treatments with the carrier receptor/drug combination without developing an allergic reaction to the carrier protein.
